(19) United States
(12) Patent Application Publication
(10) Pub. No.: US 2017/0143001 A1
(43) Pub. Date: May 25, 2017

(54) SOYBEAN OLIGOPEPTIDE WITH LOW ALLERGENICITY AND LITTLE BITTERNESS AND PREPARATION METHOD AND APPLICATION THEREOF

(71) Applicant: CHINA NATIONAL RESEARCH INSTITUTE OF FOOD AND FERMENTATION INDUSTRIES, BEIJING (CN)

(72) Inventors: MUYI CAI, BEIJING (CN); RUIZENG GU, BEIJING (CN); JUN LU, BEIJING (CN); TAO MA, BEIJING (CN); XINGCHANG PAN, BEIJING (CN); ZHE DONG, BEIJING (CN); YONG MA, BEIJING (CN); YAGUANG XU, BEIJING (CN); YONGQING MA, BEIJING (CN); ZHENTAO JIN, BEIJING (CN); LIANG CHEN, BEIJING (CN); LU LU, BEIJING (CN); WENYING LIU, BEIJING (CN); YING WEI, BEIJING (CN); HAIXIN ZHANG, BEIJING (CN); YAN LIU, BEIJING (CN); KELU CAO, BEIJING (CN); JING WANG, BEIJING (CN); GUOMING LI, BEIJING (CN); MING ZHOU, BEIJING (CN); HUI CHEN, BEIJING (CN); JIAJI LI, BEIJING (CN)

(21) Appl. No.: 15/424,715

(22) Filed: Feb. 3, 2017

Related U.S. Application Data
(63) Continuation of application No. PCT/CN2015/078137, filed on Apr. 30, 2015.

(51) Int. Cl. A23J 3/16; C12P 21/06; A23J 3/34

(52) U.S. Cl. CPC A23J 3/16; A23J 3/34; C12P 21/06

(57) ABSTRACT

The present invention provides a soybean oligopeptide with low allergenicity and little bitterness and its preparation method and application. The preparation method includes the following steps: 1) mixing a soybean protein powder with water to obtain a soybean protein solution, and then performing thermal denaturation on the soybean protein solution, to prepare a denatured protein solution; 2) adjusting pH value of the denatured protein solution to 6-9, and then adding a neutral protease and papain to conduct a first enzymolysis, to obtain a first enzymatic hydrolysate; 3) adding an alkaline protease and a flavor protease into the first enzymatic hydrolysate to conduct a second enzymolysis, and after performing enzyme inactivation, to obtain a second enzymatic hydrolysate; and 4) centrifuging the second enzymatic hydrolysate, and performing membrane filtration on centrifuged supernatant liquid, to obtain the soybean oligopeptide with low allergenicity and little bitterness.

### CROSS-REFERENCE TO RELATED APPLICATIONS

This application is a continuation of international application No. PCT/CN2015/078137 filed on Apr. 30, 2015. The content of the above identified application is incorporated herein by reference in its entirety.

### TECHNICAL FIELD

The present invention relates to a soybean oligopeptide, and particularly to a soybean oligopeptide with low allergenicity and little bitterness and preparation method and application thereof.

### BACKGROUND

Soybean proteins are plant proteins, have amino acid compositions similar to those of milk proteins and are rich in various essential amino acids, and the soybean proteins are equivalent to animal proteins in nutritive value, are closest to human amino acids in gene structure and thus are considered as the most nutritious plant proteins. However, multiple allergens are present in the soybean proteins, for example, glycinin, β-conglycinin, P34, GlymBd 28K and so on, among which glycinin and β-conglycinin are main components of in soybean proteins, adding up to about 70%; and some soybean proteins, for example, soybean trypsin inhibitor (STI), retain stable structure during conventional production processes (for example, under high temperature conditions), and are hence generally used as indicators for detecting soybean allergenic proteins. Currently, there are about 1-6% of infants who are affected by soybean allergens and thereby produce soybean allergies, such as produce respiration, skin, gastrointestinal or other symptoms, and furthermore as soybean products increase, incidence of allergies in adults is rising.

The method for deallergization of soybean proteins includes heat treatment, chemical treatment, fermentation methods, enzyme methods and the like. The heat treatment is the most common approach for deallergization of soybean allergens, and is able to alter the structure of soybean proteins and reduce allergization activity of antigenic proteins; however, it is impossible to completely eliminate allergization of the soybean proteins by heating alone due to complex structure of surface epitopes of P34 protein. The chemical treatment is mainly used for reducing activity of trypsin inhibitor by a chemical reagent, but it will inevitably produce food safety issues such as chemical residues.

The fermentation method mainly uses mould, Bacillus subtilis and other microorganisms to degrade antigenic proteins in soybean products, and although the soybean proteins can be hydrolyzed, by fermentation, into small molecular peptides with low allergenicity, it is still a question whether the hydrolyzed proteins remain necessary conformations recognizable by antibodies. For example, the China patent publication No. CN101990984A discloses a method for preparation of fermented soybean meal, which is used as feed, with high oxidation-resistance and low allergenicity, wherein Aspergillus oryzae is used for fermentation of soybean meal fermentation base stock, and although large molecular proteins are significantly degraded after fermentation, allergenicity of the fermented products is not detected, and therefore, it is unable to determine whether there are still allergenic soybean fragments in the fermented products; besides, the method fails to evaluate the taste of the fermented products. Herian etc., detected the allergenicity of five conventional soybean fermented products, including bean sprouts, acid hydrolyzed soy sauce, mould hydrolyzed soy sauce, fermented black bean and bean paste, by radioallergosorbent test (RAST), the results showed that the five soybean fermented products have roughly the same capacity to combine with serum IgE of allergic patients, thereby indicating that although soybean proteins are hydrolyzed into small molecular peptides, allergenic soybean proteins or fragments thereof are still present to some extent.

The enzyme method can hydrolyze antigenic proteins of soybeans with specific enzymes, and the effect thereof is influenced by many factors, such as type of enzyme, pretreatment before hydrolysis, and degree of hydrolysis; in particular, as the soybean proteins have a variety of allergens and complex surface epitope structures, it also poses a challenge on how to simultaneously degrade the variety of allergens to completely eliminate allergization of the soybean proteins. In addition, although the enzymolysis may effectively damage antigenic epitopes of soybean antigenic proteins, there are some concerns that the enzymolysis products acquires new allergenicities due to exposure of some linear epitopes hidden within three-dimensional protein structures or hydrophobic regions. At the same time, degradation process of the enzyme method may induce the release of bitter and astringent components from the soybean proteins, thus affecting taste and practical application of the products.

### SUMMARY

The present invention provides a soybean oligopeptide with low allergenicity and little bitterness and preparation method and application thereof, for addressing technical defects in the prior art that allergenicity of soybean proteins cannot be eliminated completely and the taste of the products is poor.

The method for preparation of a soybean oligopeptide with low allergenicity and little bitterness provided by the present invention comprises the following steps:

1) mixing a soybean protein powder with water to obtain a soybean protein solution, and performing thermal denaturation on the soybean protein solution to obtain a denatured protein solution;

2) adjusting pH value of the denatured protein solution to 6-9, and then adding a neutral protease and papain to conduct a first enzymolysis to obtain a first enzymatic hydrolysate;

3) adding an alkaline protease and a flavor protease into the first enzymatic hydrolysate to conduct a second enzymolysis, and after performing enzyme inactivation, to obtain a second enzymatic hydrolysate; and

4) centrifuging the second enzymatic hydrolysate, and performing membrane filtration on centrifuged supernatant liquid, to obtain the soybean oligopeptide with low allergenicity and little bitterness.

In the soybean protein powder used in the present invention, total content of proteins is greater than 60%, and further 60-95%, by weight; during preparation of the soybean protein solution, a mass to volume ratio of the soybean protein powder and water may be 1: (5-10), i.e., 1 kg of the soybean protein powder is mixed with 5-10 L of water for preparing the soybean protein solution. If the concentration of the soybean protein solution (the mass to volume ratio is greater than 1:5) is too high, the solution is viscous and has poor flowability, thus giving rise to decreased enzymolysis efficiency; and if the concentration (the mass to volume ratio is less than 1:10) is too low, reaction volume is too large, which will affect the following operations (for example, membrane filtration, concentration and the like), as well as raise cost accordingly.

Further, the performing thermal denaturation includes: heating the soybean protein solution to 70-90° C., maintaining this temperature and continuously stirring for 20-60 min. The thermal denaturation treatment is able to break spatial structure of the soybean proteins, thereby decreasing allergenicity of the soybean proteins; and can also solve the problem of poor flowability and viscosity of the soybean protein solution, so as to facilitate the subsequent enzymolysis.

After making a considerable amount of researches on the use of the enzyme method to completely eliminate allergenicity of the soybean proteins while inhibiting production of bitter and astringent substances in the enzymolysis products, the inventor found that a majority of proteases are incapable of completely eliminating the allergenicity of the soybean proteins and/or inhibiting the production of bitter and astringent substances in the enzymolysis products. For example, bromelain has no obvious effect on elimination of the allergenicity of the soybean proteins; the neutral protease can eliminate of the allergenicity of the soybean proteins to a certain extent, but bitter substances are present in the enzymolysis products and unable to be removed. During the researches, the inventor surprisingly founds that, only performing the first enzymolysis using a complex enzyme composed of a neutral protease and papain and then a subsequent enzymolysis (the second enzymolysis) using a complex enzyme composed of an alkaline protease and a flavor protease can more completely eliminate the allergenicity of the soybean proteins while inhibiting the production of bitter and astringent substances in the enzymolysis products.

Particularly, in the first enzymolysis of the present invention, an amount of the neutral protease is 10-100 U/g, an amount of the papain is 10-100 U/g, the first enzymolysis is conducted at 30-60° C., time of the first enzymolysis is controlled to be 1-3 h. Further, an amount ratio of the neutral protease to the papain is 1: (1-3), for example, the amount of the neutral protease is 10 U/g, while the amount of the papain is 10-30 U/g. The combination use of the neutral protease and the papain helps to eliminate the allergenicity of soybean proteins through full degradation thereof, while inhibiting the release of the bitter and astringent components, so as to improve taste of the enzymolysis products.

During the second enzymolysis of the present invention, an amount of the alkaline protease is 10-100 U/g, an amount of the flavor protease is 10-100 U/g, the second enzymolysis is conducted at 30-60° C., and time of the second enzymolysis is controlled to be 1-3 h. Further, the second enzymolysis is conducted at a pH value of 5-8, that is to say, if the pH value of the first enzymatic hydrolysate is not within the range of 5-8, it is necessary to adjust the pH value of the first enzymatic hydrolysate to 5-8 and then add the alkaline protease and the flavor protease for the second enzymolysis; and an amount ratio of the alkaline protease to the flavor protease is 1: (1-4), for example, when the amount of the alkaline protease is 10 U/g, the amount of the flavor protease is 10-40 U/g. If time of the first enzymolysis or the second enzymolysis is too short (less than 1 h), it will go against degradation of the proteins, and if the time is too long (greater than 3 h), it may lead to the production of the bitter and astringent substances.

Following the first enzymolysis, further enzymolysis with a combination of an alkaline protease and a flavor protease is conducive to further degradation of the first enzymolysis products, so as to eliminate the allergenicity of the soybean proteins, and control the release of the bitter and astringent components to improve the taste of enzymolysis products; the two enzymolysis steps may reduce the total content of both main allergenic proteins (including glycinin and β-conglycinin) and trypsin inhibitor in the soybean proteins by 99% or more. In addition, the two enzymolysis steps contribute to full degradation of the soybean proteins into oligopeptides with smaller molecular weights (for example, peptides with a molecular weight of less than 1000 Da), thus helping to improve utilization ratio of the soybean proteins .

In the present invention, amounts of the enzymes are based on the weight of the soybean protein powder, i.e., 10-100 U of the neutral protease is used when lg of the soybean protein powder is used for preparing the soybean protein solution. Further, the enzyme inactivation is performed at 110-120° C., and time of the enzyme inactivation is controlled to be 10-30min.

Further, rotation speed during the centrifuging in step 4) may be controlled at 2000-6000 r/min. The centrifuging may be performed with conventional equipment, for example, a horizontal spiral centrifuge, a tubular centrifuge and the like. In addition, the membrane filtration may be conducted using a filtration membrane with a pore diameter of 1-200 nm and further 1-50nm; during the membrane filtration, absolute pressure of the membrane filtration may be controlled at 0.2-0.4 MPa, and the temperature is controlled at 30-80° C. Membrane filtration for centrifuged supernatant liquid of the second enzymatic hydrolysate can further intercept components with large molecular weight, so as to maximize the removal of large-molecular-weight allergenic protein components in the enzymatic hydrolysate.

In the present invention, after the membrane filtration, the resulting filtrate may be decolorized and concentrated. Specifically, the decolorization may be conducted by a conventional decolorizer, for example, activated carbon powder, the mass ratio of the decolorizer to the filtrate may be (5-10):100, an temperature of the decolorization may be controlled at 70˜90° C., for example, 80° C., time of the decolorization may be 20-40 min, the decolorization may be conducted under stirring. After the decolorization, the decolorizer may be removed through a conventional method, for example, using a plate and frame filter. Further, the filtrate removal of the decolorizer may be concentrated by evaporation, for example, a double-effect falling film evaporator may be used to conduct concentration. During concentration by the evaporation, vapor pressure may be controlled at 0.1±0.02 MPa and evaporation temperature at 40-80° C. After the concentration, the volume of the concentrated solution may be reduced to ⅓-½ of the original volume. Further, sterilization and drying may be conducted after the concentration, thus preparing the soybean oligopeptide powder with low allergenicity and little bitterness. The drying, for example, may be spray-drying.

The present invention also provides a soybean oligopeptide with low allergenicity and little bitterness, which is prepared according to any one of the preparation methods described above. In the soybean oligopeptide with low allergenicity and little bitterness, content of the glycinin is less than 200 mg/kg, content of the β-conglycinin is less than 150 mg/kg, and content of the soybean trypsin inhibitor is less than 100 mg/kg; further, in the soybean oligopeptide with low allergenicity and little bitterness, the content of the glycinin is less than 125 mg/kg, the content of the β-conglycinin is less than 90 mg/kg, and the content of the soybean trypsin inhibitor is less than 50 mg/kg.

Further, in the soybean oligopeptide with low allergenicity and little bitterness, content of peptides with a molecular weight of less than 5000 Da is greater than 85% by weight, and content of peptides with a molecular weight of less than 1000 Da is greater than 60% by weight; and further, in the soybean oligopeptide with low allergenicity and little bitterness, the content of peptides with a molecular weight of less than 5000 Da is greater than 95% by weight, and the content of peptides with a molecular weight of less than 1000 Da is greater than 85% by weight.

The present invention also provides applications of the above soybean oligopeptide with low allergenicity and little bitterness in milk powder or health food. The milk powder may include infant milk powder, adult milk powder, middle- and old-aged adult milk powder and the like.

In the method of the present invention, after the thermal denaturation of the soybean proteins, four specific proteases are used to conduct the enzymolysis in two steps, not only solving the problem of incapable of completely eliminating the allergenicity of soybean proteins with various allergens and complex surface epitope structures, by reducing total content of main allergenic proteins, i.e., glycinin, β-conglycinin and soybean trypsin inhibitor, in the soybean proteins by 99% or more; in addition, the method prevents the release of bitter and astringent components from the soybean proteins, thus ensuring taste of products thereof. The method of the present invention has simple process and thus is suitable for large scale production, and the prepared soybean oligopeptide with low allergenicity and little bitterness has a wide range of applications.

### DETAILED DESCRIPTION

In order to make the purpose, technical solutions and advantages of the present invention clearer, the technical solutions of the present invention will be clearly and completely described in conjunction with the examples of the present invention, and obviously, the described examples are merely part rather than all of the examples of the present invention. Based on the examples of the present invention, all other examples obtained by one with ordinary skill in the art without creative efforts shall fall into the protection scope of the present invention.

All proteases used in the present invention were bought from Novozymes Biotechnology Co., Ltd.

### EXAMPLE 1

1. Thermal Denaturation

500 kg of a soybean protein powder with protein content of about 60% and then 4000 L of water were added into a reactor and stirred for mixing uniformly, to prepare a soybean protein solution. The soybean protein solution was heated to about 80° C., maintaining this temperature and continuously stirring for about 40 min, to prepare a denatured protein solution.

2. First Enzymolysis

The denatured protein solution was cooled to about 50° C., and pH value thereof was adjusted to about 7. A neutral protease and papain were added into the denatured protein solution, where amounts of the neutral protease and the papain were both about 50 U per gram of soybean protein powder. Remaining at the temperature of about 50° C., a first enzymolysis was performed for about 3 h, thereby preparing a first enzymatic hydrolysate.

3. Second Enzymolysis

To the first enzymatic hydrolysate obtained above was added an alkaline protease and a flavor protease, where an amount of the alkaline protease was about 50 U per gram of soybean protein powder, an amount of the flavor protease was about 100 U per gram of soybean protein powder. Remaining at the temperature of about 50° C., a second enzymolysis was performed for about 2 h. The resulting enzymatic hydrolysate was heated to 120° C. and subjected to enzyme inactivation for 20 min, thereby preparing a second enzymatic hydrolysate.

4. Centrifugation and Membrane Filtration

The second enzymatic hydrolysate was centrifuged at a rotation speed of 4000 r/min, and the centrifuged supernatant liquid was collected for later use;

The centrifuged supernatant liquid was filtered with a ceramic membrane having a pore diameter of about 50 nm, where the absolute pressure during filtration was controlled at about 0.3 MPa and the temperature at about 50° C., thereby obtaining a filtrate.

5. Decolorization, Concentration and Sterilization

To the filtrate was added an activated carbon powder in a mass ratio of 10:100 of the activated carbon powder to the filtrate. Then decolorization was performed at about 80° C. for 30 min under stirring, and after the decolorization, the activated carbon powder was removed via a plate and frame filter, to obtain a decolorized solution.

The decolorized solution was concentrated by evaporation to half of original volume thereof, where the vapor pressure was controlled at about 0.1 MPa and the evaporation temperature at about 60° C. Sterilization and spray-drying were conducted on the concentrated solution, thus preparing a soybean oligopeptide with low allergenicity and little bitterness.

6. Performing a Quality Detection and Taste Evaluation

A Glycincin ELISA Kit (from the Unibiotest Company) and a β-conglycinin ELISA Kit (from the Unibiotest Company) were used for detecting contents of glycinin and β-conglycinin in the soybean oligopeptide with low allergenicity and little bitterness, respectively, a Soy Allergens reagent kit (from the ELISASYSTEM Company) was used for detecting content of the soybean trypsin inhibitor in the soybean oligopeptide with low allergenicity and little bitterness, while a soybean protein solution without any treatment was used as a blank control. Quality detection results were shown in table 1.

The molecular weight distribution of various components in the soybean oligopeptide with low allergenicity and little bitterness, as prepared above, was detected in accordance with GB/T 22729-2008. The results were shown in table 2.

The soybean oligopeptide with low allergenicity and little bitterness prepared above was dissolved in water to prepare a solution containing 10% by weight of the soybean oligopeptide with low allergenicity and little bitterness; and establishing an evaluation group of 20 people (half men and half women) for bitterness evaluation of the solution of the soybean oligopeptide with low allergenicity and little bitterness, and the evaluation method is as follows: taking 1mL of the solution of the soybean oligopeptide with low allergenicity and little bitterness, and conducting a gradient dilution on the solution, until a bitterness was just tasted, and calculating an average bitterness value of the 20 people with the dilution multiple as the bitterness value. The results were shown in table 3.

### EXAMPLE 2

1. Thermal Denaturation

500 kg of a soybean protein powder with protein content of about 65% and then 5000 L of water were added into a reactor, and stirred for mixing uniformly, to prepare a soybean protein solution. The soybean protein solution was heated to about 90° C., maintaining this temperature and continuously stirring for about 20 min, to prepare a denatured protein solution.

2. First Enzymolysis

The denatured protein solution was cooled to about 40° C., and pH value thereof was adjusted to about 8. A neutral protease and papain were added into the denatured protein solution, where an amount of the neutral protease was about 10 U per gram of soybean protein powder, and an amount of the papain was about 30 U per gram of soybean protein powder. Remaining at the temperature of about 40° C., a first enzymolysis was performed for about 2 h, thus preparing a first enzymatic hydrolysate.

3. Second Enzymolysis

To the first enzymatic hydrolysate obtained above was added an alkaline protease and a flavor protease, where amounts of the alkaline protease and the flavor protease were both about 75 U per gram of soybean protein powder. Remaining at the temperature of about 40° C., a second enzymolysis was performed for about 3 h. The resulting enzymatic hydrolysate was heated to 110° C., performing enzyme inactivation for 30 min, to prepare a second enzymatic hydrolysate.

4. Centrifugation and Membrane Filtration

The second enzymatic hydrolysate was centrifuged at a rotation speed of 3500 r/min, and the centrifuged supernatant liquid was collected for later use;

The centrifuged supernatant liquid was filtered with a filtration membrane having a pore diameter of about 200 nm, where the absolute pressure during the filtration was controlled at about 0.4 MPa and the temperature at about 80° C., thereby obtaining a filtrate.

5. Decolorization, concentration and sterilization

To the filtrate was added an activated carbon powder in a mass ratio of 5:100 of the activated carbon powder to the filtrate. Then decolorization was conducted at about 80° C. for about 30 min under stirring, and after the decolorization, the activated carbon powder was removed via a plate and frame filter, to obtain a decolorized solution;

The decolorized solution was concentrated by evaporation to ⅓ of original volume thereof, where the vapor pressure was controlled at about 0.1 MPa and the evaporation temperature at about 80° C. Sterilization and spray-drying were conducted on the concentrated solution, thereby preparing a soybean oligopeptide with low allergenicity and little bitterness. The quality detection results, molecular weight distribution and taste evaluation results of the soybean oligopeptide with low allergenicity and little bitterness were respectively shown in table 1 to table 3.

### EXAMPLE 3

1. Thermal Denaturation

500 kg of a soybean protein powder with protein content of about 70% and then 2500 L of water were added to a reactor and stirred for mixing uniformly, to prepare a soybean protein solution. The soybean protein solution was heated to about 80° C., maintaining this temperature and continuously stirring for about 60min, to prepare a denatured protein solution.

2. First Enzymolysis

The denatured protein solution was cooled to about 60° C., and pH value thereof was adjusted to about 6. A neutral protease and papain were added into the denatured protein solution, where an amount of the neutral protease was about 50 U per gram of soybean protein powder, and an amount of the papain was about 100 U per gram of soybean protein powder. Remaining at the temperature of about 60° C., a first enzymolysis was conducted for about 1 h, thus preparing a first enzymatic hydrolysate.

3. Second Enzymolysis

To the first enzymatic hydrolysate obtained above was added an alkaline protease and a flavor protease, where an amount of the alkaline protease was about 40 U per gram of soybean protein powder, and an amount of the flavor protease was about 160 U per gram of soybean protein powder. Remaining at the temperature of about 60° C., a second enzymolysis was conducted for about lh. The resulting enzymatic hydrolysate was heated to 120° C. and subjected to enzyme inactivation for 20 min, thus preparing a second enzymatic hydrolysate.

4. Centrifugation and Membrane Filtration

The second enzymatic hydrolysate was centrifuged at a rotation speed of 4000 r/min, and the centrifuged supernatant liquid was collected for later use;

The centrifuged supernatant liquid was filtered with a filtration membrane having a pore diameter of about 50 nm, where the absolute pressure during filtration was controlled at about 0.2 MPa and the temperature at about 30° C., thereby obtaining a filtrate.

5. Decolorization, Concentration and Sterilization

To the filtrate was added an activated carbon powder in a mass ratio of 8:100 of the activated carbon powder to the filtrate. Then decolorization was conducted at about 80° C. for about 30 min under stirring, and after the decolorization, the activated carbon powder was removed via a plate and frame filter, to obtain a decolorized solution;

The decolorized solution was concentrated by evaporation to ⅓ of original volume thereof, where the vapor pressure was controlled at about 0.1 MPa and the evaporation temperature at about 60° C. Sterilization and spray-drying were conducted on the concentrated solution, thus preparing a soybean oligopeptide with low allergenicity and little bitterness. The quality detection results, molecular weight distribution and taste evaluation results of the soybean oligopeptide with low allergenicity and little bitterness were respectively shown in table 1 to table 3.

### COMPARATIVE EXAMPLE 1

The denatured protein solution prepared in Example 1 was cooled to about 40° C., and pH value thereof was adjusted to about 8. A neutral protease was added into the denatured protein solution in an amount of about 100 U per gram of soybean protein powder.

Maintaining at the temperature of about 40° C., an enzymolysis was performed for about 5 h, and the resulting enzymatic hydrolysate was centrifuged, concentrated, sterilized, and dried in sequence, in accordance with the method of Example 1, thus preparing a soybean peptide. The quality detection results and taste evaluation results thereof were respectively shown in table 1 and table 3.

### COMPARATIVE EXAMPLE 2

The denatured protein solution prepared in Example 1 was cooled to about 50° C., and pH value thereof was adjusted to about 7. Bromelain was added into the denatured protein solution in an amount of about 250 U per gram of soybean protein powder.

Maintaining at the temperature of about 50° C., an enzymolysis was performed for about 5 h, and the resulting enzymatic hydrolysate was centrifuged, concentrated, sterilized, and dried in sequence, in accordance with the method of Example 1, thus preparing a soybean peptide. The quality detection results and taste evaluation results thereof were respectively shown in table 1 and table 3.

### COMPARTIVE EXAMPLE 3

The second enzymatic hydrolysate prepared in Example 1 was directly centrifuged, concentrated, sterilized and dried in sequence to in accordance with the method of Example 1 without going through membrane filtration and decolorization, to prepare a soybean peptide. The quality detection results and taste evaluation results thereof were respectively shown in table 1 and table 3.

Quality detection results of soybean peptides in table 1

| | | | Content of |
|---|---|---|---|
| Experimental | Content of | Content of | soybean trypsin |
| example | glycinin | β-conglycinin | inhibitor |
| Blank control | 3.78 × 10⁵ mg/kg | 2.98 × 10⁵ mg/kg | 1.57 × 10⁴ mg/kg |
| Example 1 | 124.72 mg/kg | 79.95 mg/kg | 46.74 mg/kg |
| Example 2 | 56.84 mg/kg | 68.69 mg/kg | 31.97 mg/kg |
| Example 3 | 117.48 mg/kg | 85.85 mg/kg | 46.53 mg/kg |
| Comparative | 6.78 × 10⁴ mg/kg | 4.11 × 10⁴ mg/kg | 8.24 × 10³ mg/kg |
| Example 1 | | | |
| Comparative | 3.52 × 10⁴ mg/kg | 2.45 × 10⁴ mg/kg | 5.42 × 10³ mg/kg |
| Example 2 | | | |
| Comparative | | | |
| Example 3 | 6.80 × 10³ mg/kg | 5.82 × 10³ mg/kg | 708.42 mg/kg |

It can be concluded from the results of table 1:

1. In the soybean oligopeptide with low allergenicity and little bitterness prepared by the present invention, the contents of allergenic proteins, namely, glycinin, β-conglycinin and soybean trypsin inhibitor were significantly reduced, and the total content of the three proteins may be reduced by 99% by weight or more. This showed that the method of the present invention was able to completely eliminate allergenicity of soybean proteins and had excellent deallergization effect.

2. The deallergization effect of the soybean proteins was not obvious when using bromelain for treating the soybean proteins; and in the case of adopting a neutral protease for treating the soybean proteins, the allergenicity of the soybean proteins was able to be eliminated to a certain extent, but the deallergization effect was not very good.

3. The allergenic protein components of soybean were unable to be eliminated completely just through enzymolysis technology, and the soybean allergen can be eliminated to the maximum extent only using complex enzymolysis technology of the present invention in combination with specific processes such as membrane filtration and decolorization.

This showed that not arbitrary proteases or combinations thereof were able to reduce or eliminate allergenicity of soybean proteins when being used to treat the soybean proteins, and only the adoption of proteases with specific composition and specific processes (for example, pre-denaturation, stepwise enzymolysis, membrane filtration, decolorization and so on) were able to completely eliminate the allergenicity of soybean proteins.

TABLE 2
Molecular weight distribution of the soybean oligopeptide with low
allergenicity and little bitterness

| Range of molecular | | | |
|---|---|---|---|
| weight | Example 1 | Example 2 | Example 3 |
| More than 5000 | 2.34 | 1.13 | 2.23 |
| 1000-5000 | 5.20 | 10.24 | 9.69 |
| 500-1000 | 22.78 | 27.02 | 29.64 |
| 140-500 | 65.32 | 56.51 | 54.85 |
| Less than 140 | 4.22 | 5.09 | 3.49 |
| 5000 or less | 97.66 | 98.87 | 97.77 |
| 1000 or less | 92.32 | 88.62 | 87.98 |

It can be concluded from the results of table 2:

The contents of peptides with a molecular weight of less than 5000 Da in the soybean oligopeptide with low allergenicity and little bitterness prepared by the present invention were greater than 95% by weight, and the contents of peptides with a molecular weight of less than 1000 Da were greater than 85% by weight.

TABLE 3
Taste evaluation results of the soybean peptides

| Experimental examples | Average bitterness value |
|---|---|
| Example 1 | 3 |
| Example 2 | 2 |
| Example 3 | 2 |
| Comparative Example 1 | 6 |
| Comparative Example 2 | 7 |
| Comparative Example 3 | 6 |

It can be concluded from the results of table 3:

The soybean oligopeptide with low allergenicity and little bitterness prepared by the present invention had a low amount of bitter components and thus had a good flavor, showing that the method of the present invention was able to efficiently inhibit production of bitter substances in the enzymolysis products; and the adoption of proteases such as bromelain, neutral protease, etc., for processing the soybean proteins fails to efficiently avoid the release of bitter and astringent components from the soybean proteins.

At last, it should be stated that, the above examples are merely intented to illustrate rather than limit the technical solutions of the present invention; although the present invention has been described in detail in accordance with the above examples, one with ordinary skill in the art should understand, that modifications can still be made to the technical solutions recorded in the above examples, or equivalent replacements can still be made to part or all the technical features therein; and neither these modifications nor these replacements shall make essence of the corresponding technical solutions depart from the scope of the technical solutions of these examples of the present invention.

What is claimed is:

1. A method for preparation of a soybean oligopeptide with low allergenicity and little bitterness, comprising the following steps:
1) mixing a soybean protein powder with water to obtain a soybean protein solution, and performing thermal denaturation on the soybean protein solution to obtain a denatured protein solution;
2) adjusting pH value of the denatured protein solution to 6-9, and then adding a neutral protease and papain to conduct a first enzymolysis to obtain a first enzymatic hydrolysate;
3) adding an alkaline protease and a flavor protease into the first enzymatic hydrolysate to conduct a second enzymolysis, and after performing enzyme inactivation, to obtain a second enzymatic hydrolysate; and
4) centrifuging the second enzymatic hydrolysate, and performing membrane filtration on centrifuged supernatant liquid, to obtain the soybean oligopeptide with low allergenicity and little bitterness.

2. The method in accordance with claim 1, wherein a mass to volume ratio of the soybean protein powder and water is 1: (5-10).

3. The method in accordance with claim 1, wherein the performing thermal denaturation comprises: heating the soybean protein solution to 70-90° C., maintaining this temperature and continuously stirring for 20-60min.

4. The method in accordance with claim 1, wherein an amount of the neutral protease is 10-100 U/g, an amount of the papain is 10-100 U/g, the first enzymolysis is conducted at 30-60° C., and time of the first enzymolysis is controlled to be 1-3 h.

5. The method in accordance with claim 1, wherein an amount of the alkaline protease is 10-100 U/g, an amount of the flavor protease is 10-100U/g, and the second enzymolysis is conducted at 30-60° C., and time of the second enzymolysis is controlled to be 1-3 h.

6. The method in accordance with claim 1, wherein the enzyme inactivation is performed at 110-120° C., and time of the enzyme inactivation is controlled to be 10-30 min.

7. The method in accordance with claim 1, wherein the membrane filtration is performed using a filtration membrane with a pore diameter of 1-200 nm.

8. A soybean oligopeptide with low allergenicity and little bitterness prepared by the method according to claim 1, wherein in the soybean oligopeptide with low allergenicity and little bitterness, content of glycinin is less than 200 mg/kg, content of β-conglycinin is less than 150 mg/kg, and content of soybean trypsin inhibitor is less than 100 mg/kg.

9. The soybean oligopeptide with low allergenicity and little bitterness in accordance with claim 8, wherein content of peptides with a molecular weight of less than 5000 Da in the soybean oligopeptide with low allergenicity and little bitterness is greater than 85% by weight, and content of peptides with a molecular weight of less than 1000 Da is greater than 60% by weight.

10. Use of the soybean oligopeptide with low allergenicity and little bitterness in accordance with claim 8 in milk powder or health food.
